(19) United States
(12) Patent Application Publication
(10) Pub. No.: US 2011/0287529 A1
(43) Pub. Date: Nov. 24, 2011

(54) Syringe-Shaped Culture Tube and Cell Culture Apparatus Using Same

(75) Inventors: Seok Kee Hong, Seoul (KR); Si Eun Yang, Seoul (KR); Kyung Suk Kim, Seoul (KR); Hyun Soo Yoon, Seoul (KR); Tae Yong Lee, Daejeon (KR); Jin Hwa Lee, Seoul (KR)

(73) Assignee: CoreStem Co., Ltd., Chungcheongbuk-do (KR)

(21) Appl. No.: 13/120,986

(22) PCT Filed: Sep. 23, 2009
(86) PCT No.: PCT/KR2009/005427

§ 371 Date: Jul. 12, 2011

(30) Foreign Application Priority Data
Sep. 26, 2008 (KR) 1020080094821

(51) Int. Cl. C12M 1/34; C12M 3/00; C12M 1/24

(52) U.S. Cl. 435/288.1; 435/304.1; 435/298.1

(57) ABSTRACT

Disclosed are a syringe-shaped culture tube which has a wide surface area and is changeable by a user to a desired size, and a cell culture apparatus in which a plurality of the culture tubes are mounted. The cell culture apparatus allows a culture medium to constantly and smoothly contact the entire inner circumference of the culture tube and rotate by a rotation unit at a preset speed when the culture tube is filled with the culture medium. Thus, the cell culture apparatus can promote stirring and gas supply and increase the gas exchange rate. Also, it can reduce consumption of the culture medium.

The cell culture apparatus according to the present disclosure includes: the culture tube with a culture space formed therein, wherein a vent hole through which the culture medium flows in and out of the culture space is formed at one or both ends of the culture tube and the inner diameter of which gradually tapers toward the outside at one end thereof is formed; a rotation support member with a plurality of the culture tubes horizontally mounted in a longitudinal direction that rotates around a rotation shaft formed at the center; and a rotation unit for rotating the rotation support member around the rotation shaft.

### TECHNICAL FIELD

The present disclosure relates to a syringe-shaped culture tube and a cell culture apparatus using the same. More particularly, the disclosure relates to a culture tube which has a wide surface area and thus is capable of effective cell culture, and a cell culture apparatus with a plurality of the culture tubes mounted, wherein the plurality of the culture tubes are rotated at a preset speed so as to allow culture of cells in large quantity while uniformly supplying a culture medium to the culture tube so that oxygen is sufficiently supplied to the cells.

### BACKGROUND

In general, cell culture refers to the process of growing and proliferating pieces of tissue aseptically isolated from a multicellular organism in a medium containing nutrients. Culture of living cells in a culture tube is carried out for various purposes, including collection of byproducts of cellular metabolism, preparation of virus vaccines, artificial cell culture for constructing artificial organs, production of medicine through genetic modification of animal cells, breeding through fusion of plant cells, and so forth.

In case the cultured cell is an animal cell, a culture medium containing such nutrients as amino acids, saccharides, minerals, vitamins, etc. is necessary and the culture condition is complex. In contrast, the culture of plant cells is easier than that of animal cells since they photosynthesize although the growth rate is slow.

With the rapid development in biotechnology since the 1980s, the technique of culturing animal cells has become important especially in association with the biomedicine. As a result, the technique for culturing animal cells in large scale has gained importance since the mid-1980s.

For animal cell culture, a culture space in which the cells can be grown, a culture medium for supplying nutrients to the cells, various gases, and so forth are required. The culture medium and gases supplied to the culture space need to be changed at appropriate intervals in order to keep the tissue used for the cell culture fresh. Recently, culture techniques suited for the particular cells to be grown, e.g. hybridomas, embryonic stem cells, etc., are studied and developed for effective animal cell culture.

In particular, animal cells derived from human or animal tissue can be grown in suspension or adherent cultures. Typically, the cells exist in blood including hematopoietic stem cells are suspension cells that can be suspended in the culture medium, whereas tissue-derived cells such as skin, liver or lung cells, embryonic stem cells, mesenchymal stem cells, fibroblasts, epithelial-like cells, etc. are adherent cells that require a surface to be attached to.

Since the adherent cells should be attached to a solid surface while the suspension cells can be grown suspended in the culture medium, the space for cell culture is restricted and a large amount of culture medium, oxygen supply and contact surface area are required to retain cell viability. Accordingly, they are disadvantageous as compared to the suspension cells in large-scale cell culture.

Accordingly, large-scale cell culture is carried out mostly on suspension cells, and effective methods or systems for large-scale culture of adherent cells are yet to be developed.

### DETAILED DESCRIPTION OF THE INVENTION

The present disclosure is directed to providing a syringe-shaped culture tube which has a wide contact surface area and is changeable by a user to a desired size.

The present disclosure is also directed to providing a cell culture apparatus in which a plurality of the culture tubes are mounted, thus allowing easy culturing of cells in large quantity. The cell culture apparatus allows a culture medium to constantly and smoothly rotate by a rotation unit at a preset speed when the culture tube is filled with the culture medium. Thus, the cell culture apparatus can promote oxygen supply and increase the gas exchange rate. Also, it can reduce consumption of the culture medium.

In one general aspect, the present disclosure provides a culture tube with a culture space formed therein, wherein a vent hole through which a culture medium flows in and out of the culture space is formed at one or both ends of the culture tube and the inner diameter of which gradually tapers toward the outside at one end where the vent hole is formed.

The culture tube may have a circular, oval or polygonal cross section.

The vent hole may be formed at the center of the end of the culture tube.

The culture tube may be made of a transparent material.

In another general aspect, the present disclosure provides a cell culture apparatus including: a culture tube with a culture space formed therein, wherein a vent hole through which a culture medium flows in and out of the culture space is formed at one or both ends of the culture tube and the inner diameter of which gradually tapers toward the outside at one end where the vent hole is formed; a rotation support member with a plurality of the culture tubes horizontally mounted in a longitudinal direction that rotates around a rotation shaft; and a rotation unit for rotating the rotation support member around the rotation shaft.

The cell culture apparatus may further include a drum-shaped body accommodating the rotation support member with the plurality of the culture tubes mounted and having a cover or an opening hole that can be opened and closed at one side for easy flow in and out of the culture tube.

The rotation unit may rotate the rotation support member through magnetic rotation by means of a magnetic coupler or by driving a motor.

The cell culture apparatus may further include a heating unit outside the rotation support member or the drum-shaped body to maintain the temperature inside the culture tube at a preset temperature.

The heating unit may include a heating jacket with a space therein, and a heating member supplying heat to a heating medium accommodated in the heating jacket.

The drum-shaped body may be equipped with at least one of a temperature sensor, a humidity sensor and a gas sensor.

The cell culture apparatus may further include an angle adjustment unit provided at one side of the drum-shaped body so as to collect the culture medium from the plurality of the culture tubes at the same time by tilting the drum-shaped body to the other side so that the culture medium flows out through the vent hole, and a collection member at the other side of the drum-shaped body so as to collect the outflowing culture medium.

The collection member may include a funnel member one end of which is connected to the other side of the drum-shaped body and the outer diameter of which gradually tapers toward the outside, and a collection bucket which is connected to the other end of the funnel member and receives the culture medium passing through the funnel member.

The angle adjustment unit may include a cylinder rod operated by hydraulic pressure.

The culture tube may have a protrusion at an outer circumference so that it does not get loose from the rotation support member when tilted by the angle adjustment unit.

The rotation support member may be at least one rotation support plate installed approximately perpendicularly to the rotation shaft and having a plurality of mounting holes through which the plurality of the culture tubes can be horizontally mounted in a longitudinal direction.

The cell culture apparatus may be one for culturing adherent cells.

The rotation support member or the drum-shaped body may be made of a transparent material.

Hereinafter, exemplary embodiments of a culture tube and a cell culture apparatus according to the present disclosure will be described in detail with reference to the accompanying drawings.

FIG. 1 is a perspective view showing a culture tube according to an embodiment of the present disclosure, FIG. 2a is a perspective view showing a culture tube according to another embodiment of the present disclosure, and FIG. 2b shows a state wherein a culture medium is filled in the culture tube shown in FIG. 2b.

First, referring to FIGS. 1 and 2, a culture tube 10, 20 according to a specific embodiment of the present disclosure has a shape of a syringe, i.e. a cylinder, with a culture space 12, 22 formed therein, wherein a vent hole 14, 24a, 24b through which a culture medium flows in and out of the culture space 12, 22 is formed and the inner diameter of which gradually tapers toward the outside at one end where the vent hole 14, 24a, 24b is formed.

The vent hole 14 may be formed at one or both ends of the culture tube 10 along a longitudinal direction. As shown in FIG. 1, the vent hole 14 may be formed at one end and the other end may be blocked. Alternatively, as shown in FIG. 2, the vent hole 24a, 24b may be formed at both ends.

According to a specific embodiment of the present disclosure, the one end of the culture tube 10, 20 where the vent hole 14, 24a, 24b is formed is formed to have a beveled portion 16, 26a, 26b such that its inner diameter gradually tapers toward the outside. This ensures a smooth outflow of the culture medium.

The culture tube 10, 20 may have a circular, oval or polygonal cross section. Specifically, it may have a circular cross section such that the culture space 12, 22 has no edge and has a wide contact surface area.

The culture tube 10, 20 may be made of a transparent material to allow easy observation of the growth state of cells in the culture space. Such a material may be reinforced glass or a plastic material such as polypropylene, polyethylene, etc.

Since the culture tube 10, 20 according to the present disclosure is horizontally mounted in a longitudinal direction in a cell culture apparatus 100, as shown in FIG. 2a, the culture medium in the culture tube 20 is filled up to the lowest height of the vent hole 24a, 24b formed at one or both ends. As such, since the culture tube 20 according to the present disclosure is not completely filled with the culture medium but only approximately 15-20% is filled with the culture medium, the consumption of the culture medium can be reduced. The amount of the culture medium filled in the culture tube 20 may be adjusted by varying the size and position of the vent hole 24a, 24b. Specifically, the vent hole 24a, 24b may be formed approximately at the center of the culture tube 20 along the longitudinal direction without being biased to either end.

The culture tube 10, 20 has a protrusion 18, 28 at an outer circumference of the culture tube 10, 20, which will be described later.

FIGS. 3 and 4 are a perspective view and a side view showing a cell culture apparatus according to an embodiment of the present disclosure, FIG. 5 is a perspective view showing a state wherein a plurality of culture tubes are mounted in a rotation support member of the cell culture apparatus shown in FIG. 3, and FIG. 6 shows an operation whereby a culture medium is collected from a plurality of culture tubes at the same time by an angle adjustment unit and a collection member of the cell culture apparatus shown in FIG. 3.

FIGS. 7a and 7b show the flow of a culture medium in a culture tube by a rotation unit in the cell culture apparatus shown in FIG. 3, and FIGS. 9a and 9b compare the performance of the cell culture apparatus shown in FIG. 3 with an existing cell culture apparatus.

Referring to FIGS. 3 and 4, a cell culture apparatus 100 according to a specific embodiment of the present disclosure comprises a culture tube 10 in which cells are cultured, a rotation support member 110 with a plurality of the culture tubes 10 mounted that rotates around a rotation shaft 112 formed at the center, and a rotation unit 120 for rotating the rotation support member 110.

The cell culture apparatus 100 may further comprise a drum-shaped body 130 accommodating the rotation support member 110 with the plurality of the culture tubes 10 mounted and having a cover 145 or an opening hole 132 that can be opened and closed at one side for easy flow in and out of the culture tube 10.

Since the configuration of the culture tube 10 is substantially the same as that of the culture tube 10 shown in FIG. 1, a detailed description thereof will be omitted.

The rotation support member 110 allows the plurality of the culture tubes 10 to be horizontally mounted in a longitudinal direction, and may be at least one rotation support plate 118 installed approximately perpendicularly to the rotation shaft 112 and having a plurality of mounting holes 116 through which the plurality of the culture tubes 10 are inserted.

Specifically, as shown in FIG. 5, the rotation support member 110 may be configured such that a pair of rotation support plates 118 having a predetermined thickness are disposed with a spacing and the plurality of the culture tubes 10 are inserted to penetrate therethrough, but without being limited thereto. That is to say, the rotation support member 110 may be configured in various manners as long as the culture tubes 10 may be stably mounted horizontally the a longitudinal direction.

The culture tube 10 mounted in the rotation support member 110 should be inserted and mounted such that the culture tube 10 does not rotate on its own axis with respect to the rotation support member 110 while the rotation support member 110 is rotated by the rotation unit 120 as will be described later. If the culture tube 10 rotates on its own axis as the rotation support member 110 rotates, the culture medium in the culture tube 10 cannot contact the entire inner circumference of the culture tube 10 uniformly.

The rotation unit 120 rotates the rotation support member 110, so that the culture medium in the culture tube 10 mounted in the rotation support member 110 flows while rotating on the inner circumference of the culture tube 10. Specifically, it may be rotated through magnetic rotation by means of a magnetic coupler 114.

As shown in the figure, the magnetic rotation type rotation unit 120 using the magnetic coupler 114 is configured such that the magnetic coupler 114 is spaced apart from the rotation shaft 112 to rotate the rotation support member 110 by means of magnetic friction. Specifically, two magnetic couplers (magnetic discs) 114 comprising two opposing permanent magnets with opposite polarity may be used. A detailed description thereof will be omitted since it is well known in the related art.

Alternatively, the rotation unit 120 may be of a motor-driven type using a motor that is connected to the rotation shaft 112 and is capable of rotating the rotation shaft 112 and a pulley connected thereto. In addition, it may be any one capable of rotating the rotation support member 110.

As described, the rotation unit 120 constantly rotates the culture tube 10 filled with a preset amount of the culture medium 1 at a constant speed, so that the culture medium 1 may flow on the entire inner circumference of the cylindrical culture tube 10, thus ensuring sufficient oxygen supply. As a result, the cells can be grown efficiently.

The drum-shaped body 130 is a cylindrical shape and has a space therein in which the rotation support member 110 is accommodated.

The drum-shaped body 130 may be configured such that both ends of the rotation shaft 112 of the rotation support member 110 can penetrate slidably therethrough. In this case, the drum-shaped body 130 does not rotate even when the rotation support member 110 is rotated by the rotation unit. Of course, the drum-shaped body 130 and the rotation support member 110 may be configured to rotate around the rotation shaft 112, if necessary.

The drum-shaped body 130 may have the opening hole 132 for easy flow in and out of the culture tube 10. Alternatively, it may be configured as a door-type structure that is opened and closed.

The drum-shaped body 130 may be equipped with a gas sensor such as a CO₂ sensor and an O₂ sensor (not shown), a temperature sensor, a humidity sensor, etc. so as to allow monitoring of the state inside the body 130.

A heating unit 160 may be installed outside the drum-shaped body 130 to maintain the temperature inside the culture tube 10 at a preset temperature in order to facilitate and stabilize cell culturing.

The heating unit 160 comprises a heating jacket 162 installed below the drum-shaped body 130 and having a space therein, and a heating member 166 supplying heat to a heating medium 164 accommodated in the heating jacket 162. The heating member 166 may be, for example, a heating coil capable of providing heat using electricity.

The heating unit 160 may be configured such that an upper portion facing the drum-shaped body 130 has a semi-arch shape so as to provide a large contact surface area and ensure effective supply of heat to the drum-shaped body 130. It may be fixed to or separated from the drum-shaped body 130.

The heating jacket 162 may be equipped with a temperature sensor 170 such as a thermoscope which is capable of detecting the temperature of the heating medium 164 inside the heating jacket 162.

In an embodiment of the present disclosure, an angle adjustment unit 150 may be provided at one side of the drum-shaped body 130 so as to collect the culture medium easily from the plurality of the culture tubes 10 at the same time by tilting the drum-shaped body 130 to the other side so that the culture medium flows out through the vent hole.

As shown in FIG. 6, the angle adjustment unit 150 tilts the rotation support member 110 and the drum-shaped body 130 to the other side so that the culture medium in the culture tubes 10 flows downward at the same time. In an embodiment of the present disclosure, a cylinder rod 152 the length of which is varied by a hydraulic pressure may be installed below the drum-shaped body 130, such that as the cylinder rod 152 is extended by the hydraulic pressure, one side of the drum-shaped body 130 moves relatively upward and the other side of the drum-shaped body 130 becomes lower than the one side, so that the culture tubes 10 are tiled to the other side and the culture medium is flown out through the vent holes 14, 23a, 23b at the other side at the same time.

Although the cylinder rod 152 is used for the angle adjustment unit 150 in an embodiment of the present disclosure, any configuration capable of tilting the drum-shaped body 130 to the other side so as to allow the culture medium in the culture tubes 10 to flow down at the same time may be employed, including using an actuator.

The culture tube 10 may have a protrusion 18, 28 formed at an outer circumference of one side so that the culture tube 10 can be inserted into a mounting hole 116 of the rotation support member 110 at a fixed position and the culture tube 10 does not get loose from the rotation support member 110 when the rotation support member 110 is tilted by the angle adjustment unit 150.

Further, the drum-shaped body 130 may have a collection member 140 at the other side so as to collect the culture medium flowing out of the plurality of the culture tubes 10 when the drum-shaped body 130 and the rotation support member 110 are tilted to the other side by the angle adjustment unit 150.

The collection member 140 comprises a funnel member 142 one end of which is connected to the other side of the drum-shaped body 130 and the outer diameter of which gradually tapers toward the outside, and a collection bucket 144 which is connected to the other end of the funnel member 142 and receives the culture medium passing through a discharge hole 143a formed on the funnel member 142.

Depending on working environment and workspace, the funnel member 142 and the collection bucket 144 may be connected via a discharge line 143, and the discharge hole of the funnel member 142 connected to the discharge line 143 may be formed at the center or lower portion of the drum-shaped body 130.

The collection bucket 144 may have a cover 145 to easily discharge the culture medium. Alternatively, the collection bucket 144 may be configured to be separable so that the culture medium can be discharged at once.

Since the culture tube 10 is changeable by a user to a desired size, the usable area can be increased. Furthermore, since the plurality of the culture tubes 10 can be independently mounted in the cell culture apparatus 100, various types of cells can be cultured and isolated at the same time using the single cell culture apparatus 100.

The culture tube 10, the rotation support member 110 or the drum-shaped body 130 may be made of a transparent material such as transparent reinforced acrylics so as to allow monitoring of the cell culture state.

Since the cell culture apparatus 100 according to the present disclosure allows the culture medium to contact the entire inner circumference of the culture tube 10, it is suitable to culture the adherent cells.

Whereas the suspension cells remain in the culture medium while being stirred by rotation, the adherent cells are attached to the inner circumference of the culture tube 10 and grow while forming a monolayer. Thus, by seeding the cells in the tube-shaped culture tube 10 and rotating the culture tube 10 around the rotation shaft in the longitudinal direction, the culture medium containing the cells is allowed to flow on the inner surface of the culture tube 10 as it rotates. In other words, by constantly and smoothly stirring the culture medium at a constant speed (approximately 1 to 2 rpm), the contact surface area of the culture medium can be increased for the same volume. As a result, oxygen supply and carbon dioxide gas exchange can be facilitated through the thin film of the culture medium covering the cells, which is not immersed in the culture medium.

That is to say, since the space for cell culture is restricted for the adherent cells, a large amount of culture medium, oxygen supply and contact surface area are required to retain cell viability. As described, by rotating the culture tube 10 with 15-20% filled with the culture medium, the cell adhesion area can be increased as compared to the existing T-flask.

Since the cells alternatingly contact with the culture medium and the air while the culture tube 10 is rotated, they can be adequately supplied with oxygen.

FIGS. 7a and 7b show the flow of the culture medium in the culture tube 10 by the rotation unit. Referring to the figures, when the rotation support member 110 is rotated while the plurality of the culture tubes 10 are horizontally inserted in the rotation support member 110 in the longitudinal direction, the culture medium contained in the culture tube 10 flows slowly on the inner circumference of the culture tube 10.

As such, since the culture medium can contact the entire inner circumference of the culture tube 10 even when the culture tube 10 is not completely filled with the culture medium but only about 15-20% is filled with the culture medium, the consumption of the culture medium can be reduced.

In other words, as for the cell culture apparatus 100 according to the present disclosure, since some of the cells attached to the inner circumference of the culture tube 10 are immersed in the culture medium and the remaining are exposed to the culture space and the cells are alternatingly contacted to the culture medium by the rotation unit, the consumption of the culture medium can be reduced as compared to the existing cell culture apparatus using a culture flask where all of the cells are immersed in the culture medium.

In addition, the culture medium in the culture tube 10 flows while contacting the entire inner circumference of the culture tube 10 as the culture tube 10 is rotated by the rotation unit, solubility of air or oxygen in the culture medium can be increased and sufficient oxygen supply can be ensured. Thus, a more stable glucose metabolism of the cultured cells can be ensured when compared to the existing cell culture apparatus.

At the other side of the drum-shaped body 130, the collection member 140 may be equipped so as to collect the culture medium flowing out of the plurality of the culture tubes 10 when the drum-shaped body 130 and the rotation support member 110 are tilted to the other side by the angle adjustment unit 150.

### BRIEF DESCRIPTION OF THE DRAWINGS

FIG. 1 is a perspective view showing a culture tube according to an embodiment of the present disclosure;

FIG. 2a is a perspective view showing a culture tube according to another embodiment of the present disclosure;

FIG. 2b shows a state wherein a culture medium is filled in the culture tube shown in FIG. 2b;

FIGS. 3 and 4 are a perspective view and a side view showing a cell culture apparatus according to an embodiment of the present disclosure;

FIG. 5 is a perspective view showing a state wherein a plurality of culture tubes are mounted in a rotation support member of the cell culture apparatus shown in FIG. 3;

FIG. 6 shows an operation whereby a culture medium is collected from a plurality of culture tubes at the same time by an angle adjustment unit and a collection member of the cell culture apparatus shown in FIG. 3;

FIGS. 7a and 7b show the flow of a culture medium in a culture tube by a rotation unit in the cell culture apparatus shown in FIG. 3; and

FIGS. 8a and 8b compare the performance of the cell culture apparatus shown in FIG. 3 with an existing cell culture apparatus.

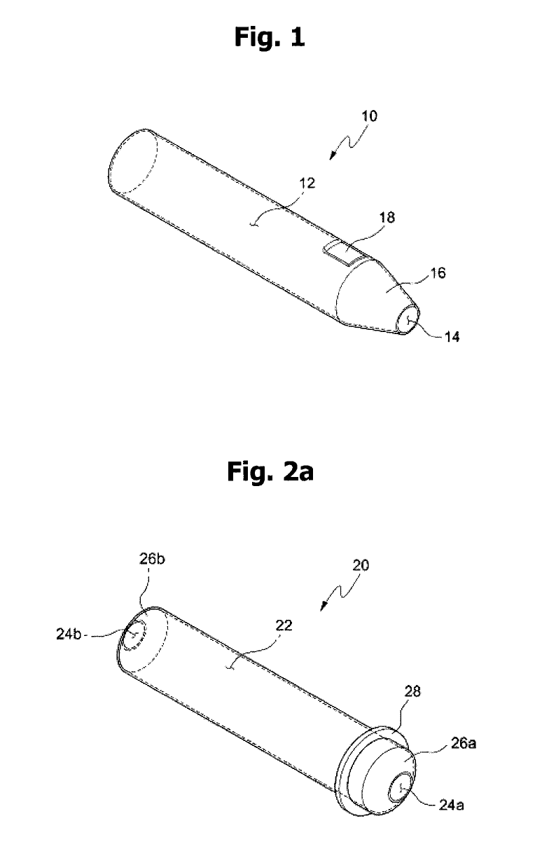

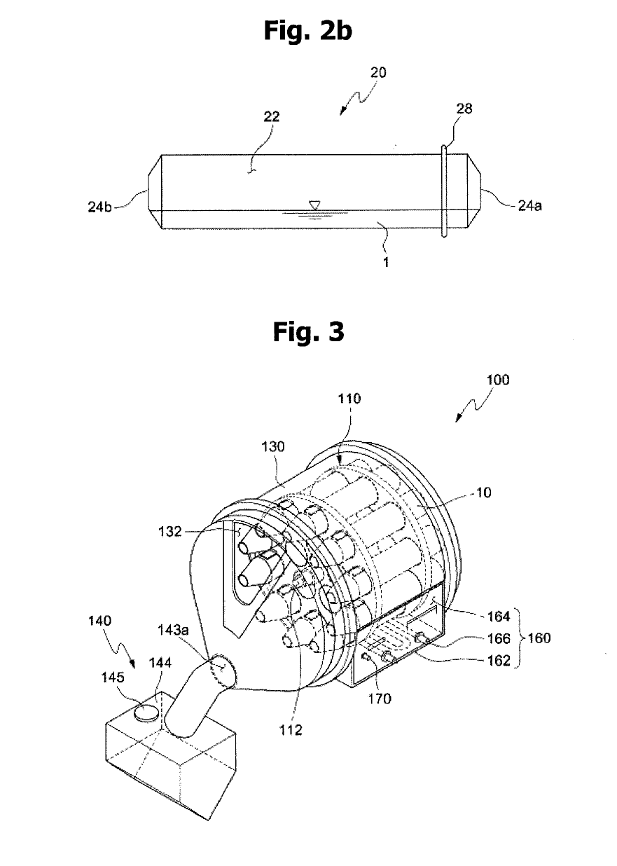

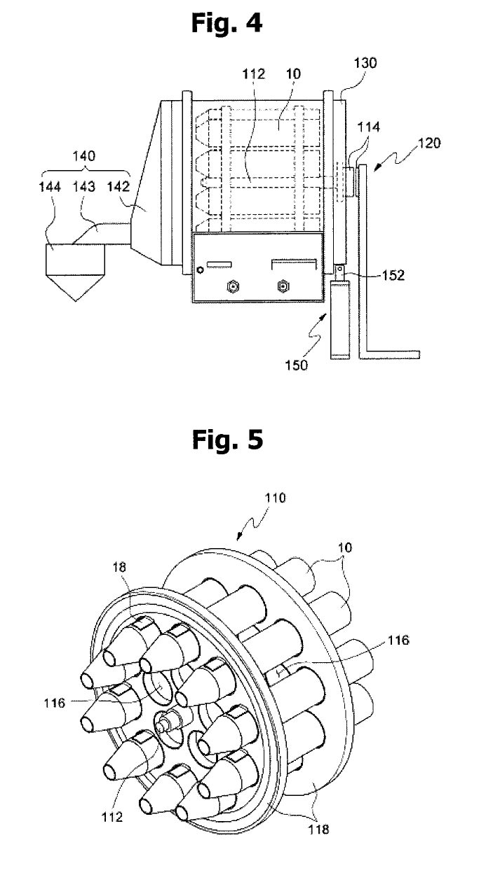

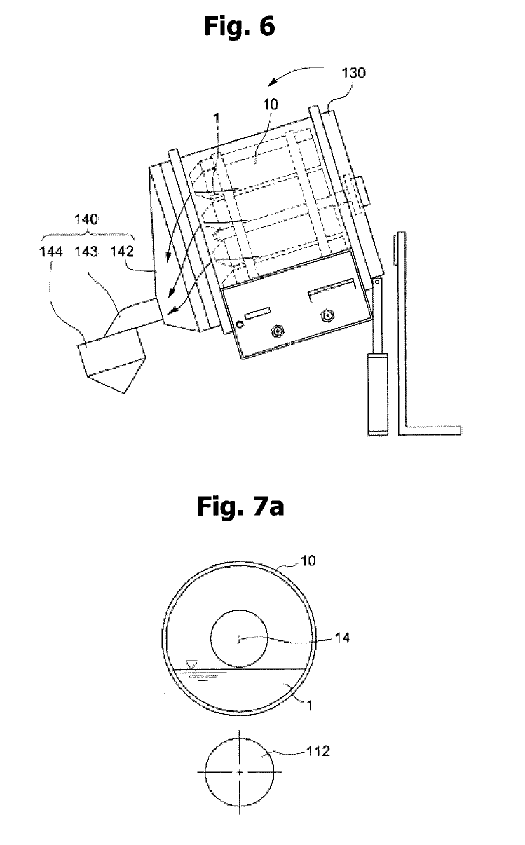

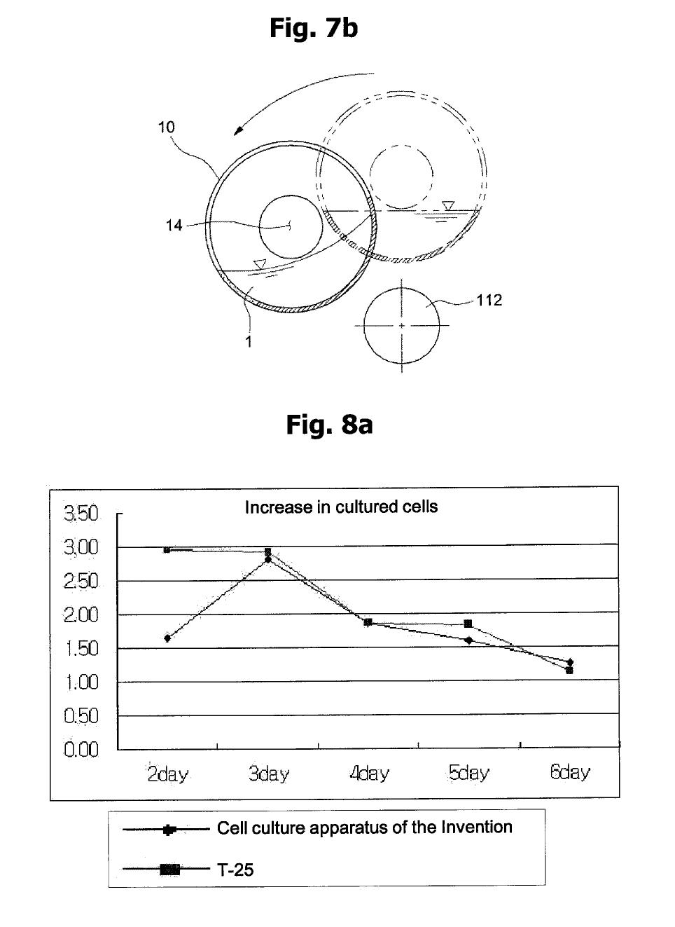

<Description of reference numerals in the drawings>

| | |
|---|---|
| 10, 20: culture tube | 12, 22: culture space |
| 14, 24a, 24b: vent hole | 16, 26a, 26b: beveled portion |
| 18, 28: protrusion | 100: cell culture apparatus |
| 110: rotation support member | 112: rotation shaft |
| 114: magnetic coupler | 116: mounting hole |
| 118: rotation support plate | 130: drum-shaped body |
| 132: opening hole | 140: collection member |
| 142: funnel member | 143: discharge line |
| 144: collection bucket | 145: cover |
| 150: angle adjustment unit | 152: cylinder rod |
| 160: heating unit | 162: heating jacket |
| 164: heating medium | 166: heating member |
| 170: temperature sensor | |

### EXAMPLES

Glucose metabolism of cells cultured in the cell culture apparatus 100 according to the present disclosure was studied as follows.

For the test, articular cartilage cells (AC cells) isolated from the human knee joint were used.

Cryopreserved AC cells from the 8th passage were thawed and seeded in a cell culture tube at a low density of 850 cells/cm² and cultured for 9 days without changing the culture medium.

During the culturing, the condition inside the cell culture apparatus was maintained at CO₂ 6.5% and 37° C. The rotation speed of the culture tube was maintained at 1 rpm.

For comparison, the same cells were seeded in a T-25 flask at the same density and cultured statically under the same condition of CO₂ 6.5% and 37° C. in a CO₂ incubator (Heraus).

During the culturing, the number of the AC cells was counted every day and glucose level was also measured every day using a glucometer.

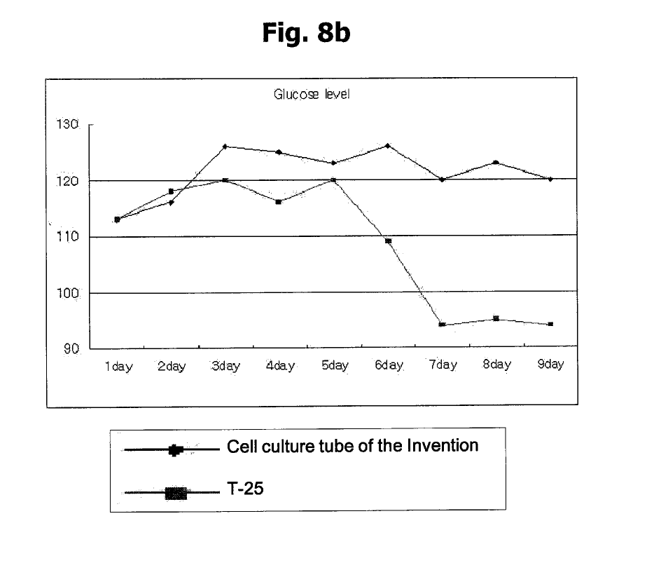

The increase of the cell number was similar in the two groups as shown in FIG. 8a, but the glucose level was higher for the cell culture apparatus according to the present disclosure than the existing T-25 flask as shown in FIG. 8b.

The better energy metabolism efficiency of the cell culture apparatus according to the present disclosure is due to the reduced decrease of glucose during the culturing since oxygen is sufficiently supplied to the cell culture tube.

Glucose is commonly used as an energy source in the culture medium for growth of cells. Under sufficient oxygen supply, it is metabolized to pyruvate by glycolysis and then oxidized to carbon dioxide and water via the citric acid cycle.

However, if the oxygen supply is not insufficient, the glycolyzed pyruvate undergoes lactate fermentation, failing to enter the citric acid cycle.

In this case, only the glycolysis process is repeated and the citric acid cycle does not occur normally, resulting in accumulation of lactate (lactic acid).

The decrease of the glucose level with the same cell growth rate means that the citric acid cycle of the cultured cells is not operated properly due to the lack of oxygen. Thus, it can be seen that the cell culture apparatus according to the present disclosure is advantageous over the static culturing using the existing flask in that the glucose metabolism can occur normally.

That is to say, since the cell culture apparatus according to the present disclosure allows the cells to attach throughout the entire inner circumference of the culture tube, the space for the cells to grow is maximized. Further, the cells can be sufficiently supplied with oxygen since they are alternatingly contacted with the culture medium and the air while the culture tube is rotated by the rotation unit.

While the present disclosure has been described with respect to the specific embodiments, it will be apparent to those skilled in the art that various changes and modifications may be made without departing from the spirit and scope of the disclosure as defined in the following claims.

As described above, the syringe-shaped culture tube and the cell culture apparatus using the same according to the present disclosure provide the following advantageous effects.

Firstly, since the culture tube has a wide contact surface area, a large number of adherent cells can be cultured. In addition, since the size of the culture tube is changeable by a user to a desired size, the efficiency of the culture tube can be maximized.

Secondly, a large quantity of cells can be cultured at the same time by using a plurality of the culture tubes. Since the plurality of the culture tubes can be independently mounted in the cell culture apparatus, various types of cells can be cultured using the single cell culture apparatus.

Thirdly, energy metabolism efficiency may be improved since oxygen can be sufficiently supplied by constantly and smoothly stirring the culture medium in the culture tube by means of the rotation unit.

Fourthly, since the culture medium partly filled in the culture tube flows on the inner surface of the culture tube as it rotates, a thin film of the culture medium is formed to cover the cells not immersed in the culture medium. As a result, the contact surface area with the culture medium is increased for the same volume of the culture medium, resulting in efficient oxygen supply and carbon dioxide discharge, and hence increased gas exchange rate.

Fifthly, since the culture tube rotates as it is partly filled with the culture medium, some of the cells attached to the culture tube are immersed in the culture medium while the remaining are exposed to the culture space of the culture tube. Thus, the consumption of the culture medium can be reduced as compared to the existing flask culture wherein all the cells are immersed in the culture medium.

What is claimed is:

1. A culture tube with a culture space formed therein, wherein a vent hole through which a culture medium flows in and out of the culture space is formed at one or both ends of the culture tube and the inner diameter of which gradually tapers toward the outside at one end where the vent hole is formed.

2. The culture tube according to claim 1, wherein the culture tube has a circular, oval or polygonal cross section.

3. The culture tube according to claim 1, wherein the vent hole is formed at the center of the end of the culture tube.

4. The culture tube according to claim 1, wherein the culture tube is made of a transparent material.

5. A cell culture apparatus comprising:
a culture tube with a culture space formed therein, wherein a vent hole through which a culture medium flows in and out of the culture space is formed at one or both ends of the culture tube and the inner diameter of which gradually tapers toward the outside at one end where the vent hole is formed;
a rotation support member with a plurality of the culture tubes horizontally mounted in a longitudinal direction that rotates around a rotation shaft; and
a rotation unit for rotating the rotation support member around the rotation shaft.

6. The cell culture apparatus according to claim 5, which further comprises a drum-shaped body accommodating the rotation support member with the plurality of the culture tubes mounted and having a cover or an opening hole that can be opened and closed at one side for easy flow in and out of the culture tube.

7. The cell culture apparatus according to claim 5 or 6, wherein the rotation unit rotates the rotation support member through magnetic rotation by means of a magnetic coupler or by driving a motor.

8. The cell culture apparatus according to claim 5 or 6, which further comprises a heating unit outside the rotation support member or the drum-shaped body to maintain the temperature inside the culture tube at a preset temperature.

9. The cell culture apparatus according to claim 8, wherein the heating unit comprises a heating jacket with a space therein, and a heating member supplying heat to a heating medium accommodated in the heating jacket.

10. The cell culture apparatus according to claim 6, wherein the drum-shaped body is equipped with at least one of a temperature sensor, a humidity sensor and a gas sensor.

11. The cell culture apparatus according to claim 6, which further comprises an angle adjustment unit provided at one side of the drum-shaped body so as to collect the culture medium from the plurality of the culture tubes at the same time by tilting the drum-shaped body to the other side so that the culture medium flows out through the vent hole, and a collection member at the other side of the drum-shaped body so as to collect the outflowing culture medium.

12. The cell culture apparatus according to claim 11, wherein the collection member comprises a funnel member one end of which is connected to the other side of the drum-shaped body and the outer diameter of which gradually tapers toward the outside, and a collection bucket which is connected to the other end of the funnel member and receives the culture medium passing through the funnel member.

13. The cell culture apparatus according to claim 11, wherein the angle adjustment unit comprises a cylinder rod operated by hydraulic pressure.

14. The cell culture apparatus according to claim 11, wherein the culture tube has a protrusion at an outer circumference so that it does not get loose from the rotation support member when tilted by the angle adjustment unit.

15. The cell culture apparatus according to claim 5, wherein the rotation support member is at least one rotation support plate installed approximately perpendicularly to the rotation shaft and having a plurality of mounting holes through which the plurality of the culture tubes can be horizontally mounted in a longitudinal direction.

16. The cell culture apparatus according to claim 5 or 6, which is for culturing adherent cells.

17. The cell culture apparatus according to claim 5 or 6, wherein the rotation support member or the drum-shaped body is made of a transparent material.
